(19) United States
(12) Patent Application Publication
(10) Pub. No.: US 2022/0386864 A1
(43) Pub. Date: Dec. 8, 2022

(54) OCT APPARATUS AND NON-TRANSITORY COMPUTER-READABLE STORAGE MEDIUM

(71) Applicant: NIDEK CO., LTD., Gamagori (JP)

(72) Inventor: Yukihiro HIGUCHI, Gamagori (JP)

(73) Assignee: NIDEK CO., LTD., Gamagori (JP)

(21) Appl. No.: 17/829,815

(22) Filed: Jun. 1, 2022

(30) Foreign Application Priority Data
Jun. 3, 2021 (JP) 2021-093706

(51) Int. Cl. A61B 3/10; A61B 3/14; A61B 3/00

(52) U.S. Cl. CPC A61B 3/102; A61B 3/14; A61B 3/0058

(57) ABSTRACT

An OCT apparatus processes an OCT signal based on reference light and measurement light with which a subject eye is irradiated to capture a tomographic image of a tissue of the subject eye. The OCT apparatus includes a controller. The controller captures a three-dimensional tomographic image of the tissue by irradiating a two-dimensional measurement region, which expands in a direction intersecting an optical axis of the measurement light, with the measurement light. The controller extracts a two-dimensional tomographic image from the three-dimensional tomographic image to display the two-dimensional tomographic image on a display unit. The controller sets an additional imaging position of a tomographic image in a state where the two-dimensional tomographic image is displayed on the display unit. The controller performs additional capturing of a tomographic image by irradiating the set additional imaging position with the measurement light.

### CROSS-REFERENCE TO RELATED APPLICATIONS

This application claims priority from Japanese Patent Application No. 2021-093706 filed on Jun. 3,2021, the entire subject-matter of which is incorporated herein by reference.

### TECHNICAL FIELD

The present disclosure relates to an OCT apparatus that captures a tomographic image of a tissue of a subject on the basis of the principles of optical coherence tomography (OCT), and a non-transitory computer-readable storage medium that stores an imaging control program executed by the OCT apparatus.

### BACKGROUND ART

A technique of capturing a tomographic image of a subject (for example, a subject eye) on the basis of the principles of OCT is known. For example, an OCT data processing apparatus disclosed in JP-A-2019-150532 includes a control unit which sets one of a plurality of types of line patterns for a two-dimensional measurement region in which a three-dimensional tomographic image of a subject eye is obtained. The control unit extracts a two-dimensional tomographic image in a line of the set line pattern from the three-dimensional tomographic image. In this case, a user can check the extraction result of the two-dimensional tomographic image corresponding to various line patterns on the basis of data of the three-dimensional tomographic image already captured.

In a case where a two-dimensional tomographic image is arbitrarily extracted from a three-dimensional tomographic image, not all pixels of the extracted two-dimensional tomographic image always match pixels forming the three-dimensional tomographic image. That is, the pixels forming the three-dimensional tomographic image may not be present at positions where the pixels forming the two-dimensional tomographic image are extracted. In this case, it is conceivable to perform a process of interpolating pixel values of the pixels forming the two-dimensional tomographic image on the basis of pixel values of neighboring pixels. However, when a process such as interpolation is performed, pixel information is often inaccurate compared with a case where the same position is scanned with measurement light to capture a two-dimensional tomographic image. The time required for capturing a three-dimensional tomographic image is longer than the time required for capturing a two-dimensional tomographic image. The longer the imaging time, the more likely the image quality will deteriorate due to the influences of fatigue, dryness, blinking, and the like of the eyes. Even in a case where the number of two-dimensional tomographic images forming a three-dimensional tomographic image is reduced in order to shorten the imaging time of the three-dimensional tomographic image, the image quality also deteriorates. As described above, in the method of extracting a two-dimensional tomographic image from a three-dimensional tomographic image, a two-dimensional tomographic image with good image quality may not be extracted. Therefore, it is more useful to be able to present, to a user, a high-quality image at a position that the user wants to check in detail in a range in which a three-dimensional tomographic image is captured.

### SUMMARY OF INVENTION

A typical object of the present disclosure is to provide an OCT apparatus and a non-transitory computer-readable storage medium storing an imaging control program capable of more appropriately presenting an image at a position where a user wants to check in detail in a range in which a three-dimensional tomographic image is captured.

A first aspect of the present disclosure is an OCT apparatus that processes an OCT signal based on reference light and measurement light with which a subject eye is irradiated to capture a tomographic image of a tissue of the subject eye, the OCT apparatus including a controller configured to:

capture a three-dimensional tomographic image of the tissue by irradiating a two-dimensional measurement region, which expands in a direction intersecting an optical axis of the measurement light, with the measurement light;

extract a two-dimensional tomographic image from the three-dimensional tomographic image to display the two-dimensional tomographic image on a display unit;

set an additional imaging position of a tomographic image in a state where the two-dimensional tomographic image is displayed on the display unit; and

perform additional capturing of a tomographic image by irradiating the set additional imaging position with the measurement light.

A second aspect of the present disclosure is a non-transitory computer-readable storage medium storing an imaging control program executed by an OCT apparatus that processes an OCT signal based on reference light and measurement light with which a subject eye is irradiated to capture a tomographic image of a tissue of the subject eye, the imaging control program including instructions which, when the imaging control program is executed by a controller of the OCT apparatus, cause the OCT apparatus to perform:

a first imaging step of capturing a three-dimensional tomographic image of the tissue by irradiating a two-dimensional measurement region, which expands in a direction intersecting an optical axis of the measurement light, with the measurement light;

an extraction display step of extracting a two-dimensional tomographic image from the three-dimensional tomographic image to display the two-dimensional tomographic image on a display unit;

an additional imaging position setting step of setting an additional imaging position of a tomographic image in a state where the two-dimensional tomographic image is displayed on the display unit; and

a second imaging step of performing additional capturing of a tomographic image by irradiating the set additional imaging position with the measurement light.

According to the OCT apparatus and the non-transitory computer-readable storage medium, it is possible to more appropriately present a user an image at a position where the user wants to check in detail in a range in which a three-dimensional tomographic image is captured.

### BRIEF DESCRIPTION OF DRAWINGS

FIG. 1 is a block diagram showing a schematic configuration of an OCT apparatus 1.

FIG. 2 is a diagram showing an example of a three-dimensional tomographic image 2 and an Enface image 3.

FIGS. 3A and 3B are flowcharts showing an imaging control process executed by the OCT apparatus 1.

FIG. 4 is a flowchart showing a first imaging process executed during the imaging control process.

FIG. 5 is a diagram showing an example of a front observation image 50.

FIG. 6 is a diagram showing an example of an imaging result check screen 60 displayed on a monitor.

FIGS. 7A and 7B are flowcharts showing a check screen display process executed during the imaging control process.

FIG. 8 is a diagram showing an example of an analysis map 73.

FIG. 9 is a diagram showing an example of a certainty factor display image 74.

FIG. 10 is a diagram showing an example of a line pattern setting screen 90.

### DESCRIPTION OF EMBODIMENTS

### Outline

An OCT apparatus exemplified in the present disclosure captures a tomographic image of a tissue of a subject eye by processing an OCT signal based on reference light and measurement light with which the subject eye is irradiated. The OCT apparatus includes an OCT light source, a branch optical element, an irradiation optical system, a combined wave optical element, a light receiving element, and a controller. The OCT light source emits light (OCT light). The branch optical element branches the light emitted from the OCT light source into measurement light and reference light. The irradiation optical system irradiates a tissue with the measurement light branched by the branch optical element. The combined wave optical element combines the measurement light reflected by the tissue and the reference light branched by the branch optical element to interfere with each other. The light receiving element detects an interference signal by receiving the interference light generated by the combined wave optical element.

The controller executes a first imaging step, an extraction display step, an additional imaging position setting step, and a second imaging step. In the first imaging step, the controller captures a three-dimensional tomographic image of the tissue by irradiating a two-dimensional measurement region, which expands in a direction intersecting an optical axis of the measurement light, with the measurement light. In the extraction display step, the controller extracts a two-dimensional tomographic image from the three-dimensional tomographic image to display the two-dimensional tomographic image on a display unit. In the additional imaging position setting step, the controller sets an additional imaging position of a tomographic image in a state where the two-dimensional tomographic image is displayed on the display unit. In the second imaging step, the controller performs additional capturing of a tomographic image by irradiating the set additional imaging position with the measurement light.

According to the OCT apparatus exemplified in the present disclosure, a two-dimensional tomographic image is extracted from a three-dimensional tomographic image that has already been captured and displayed on a display unit. In this state, an additional imaging position of a tomographic image is set, and a tomographic image is additionally captured at the set position. Therefore, a user can ascertain the additional imaging position after checking an internal state of the tissue within an imaging range of the three-dimensional tomographic image from the extracted two-dimensional tomographic image. The image quality of the additionally captured tomographic image is unlikely to deteriorate compared with the image quality of an image arbitrarily extracted from the three-dimensional tomographic image. Therefore, in the range in which the three-dimensional tomographic image is captured, an image at a position that the user wants to check in detail is presented to the user with more appropriate and high image quality.

In the extraction display step, the controller may extract a two-dimensional tomographic image having at least pixels inside the three-dimensional tomographic image to display the two-dimensional tomographic image on the display unit. In this case, the user can appropriately check an internal state of the tissue within the imaging range of the three-dimensional tomographic image from the extracted two-dimensional tomographic image.

The OCT apparatus may include a scanning unit. The scanning unit performs scanning with the measurement light applied to the tissue by the irradiation optical system in a two-dimensional direction intersecting the optical axis. The three-dimensional tomographic image may be obtained by performing scanning with a spot of the measurement light in a measurement region in the two-dimensional direction by the scanning unit. In this case, a three-dimensional tomographic image is appropriately obtained by the OCT apparatus.

However, a configuration of the OCT apparatus may be changed. For example, the irradiation optical system of the OCT apparatus may simultaneously irradiate a two-dimensional region on the tissue of the subject with the measurement light. In this case, the light receiving element may be a two-dimensional light receiving element that detects an interference signal in the two-dimensional region on the tissue. That is, the OCT apparatus may acquire OCT data according to the principle of so-called full-field OCT (FF-OCT). The OCT apparatus may simultaneously irradiate the tissue with the measurement light on an irradiation line extending in the one-dimensional direction and perform scanning with the measurement light in a direction intersecting the irradiation line. In this case, the light receiving element may be a one-dimensional light receiving element (for example, a line sensor) or a two-dimensional light receiving element. That is, the OCT apparatus may acquire a tomographic image according to the principle of so-called line field OCT (LF-OCT).

The controller may further execute a line pattern setting step of setting one of a plurality of types of line patterns in which at least one of disposition, the number, and a shape of the line is different from each other for the two-dimensional measurement region in which the three-dimensional tomographic image is obtained. The controller may extract a two-dimensional tomographic image in the line of the set line pattern from the three-dimensional tomographic image and display the two-dimensional tomographic image on the display unit. In this case, by setting a desirable line pattern, a state of the tissue within the imaging range of the three-dimensional tomographic image can be ascertained more appropriately.

A specific method of setting a line pattern may be selected as appropriate. For example, the controller may set one of a plurality of types of line patterns in response to an instruction input by the user. In this case, the two-dimensional tomographic image is extracted from the three-dimensional tomographic image in the pattern desired by the user. However, a pattern for extracting the two-dimensional tomographic image from the three-dimensional tomographic image may be set in advance.

The controller may further execute an additional imaging condition setting step of setting an imaging condition for the additional capturing of the tomographic image (hereinafter, referred to as “additional capturing condition” in some cases). The controller may perform the additional capturing of the tomographic image at an additional imaging position according to the set imaging conditions. In this case, a high-quality tomographic image is additionally captured under appropriate imaging conditions. Therefore, an image at a position that the user wants to check in detail is presented more appropriately.

The imaging conditions that can be set in the additional imaging condition setting step may include an imaging pattern for additionally capturing a tomographic image (hereinafter, referred to as an “additional imaging pattern”). The additional imaging pattern is a pattern that determines at least one of disposition, the number, and a shape of tomographic images to be additionally captured (for example, a pattern that determines at least one of disposition, the number, and a shape of lines that perform scanning with the measurement light for the additional capturing of the tomographic image). By setting the additional imaging pattern, an image at a position that the user wants to check in detail is additionally captured with a more appropriate pattern.

A specific method of setting an additional imaging pattern can also be selected as appropriate. For example, the controller may set one of a plurality of types of additional imaging patterns in response to an instruction input by the user. In this case, a tomographic image is additionally captured with an imaging pattern desired by the user. The additional imaging pattern may include a pattern for capturing one or more tomographic images. The additional imaging pattern may include a pattern for capturing a three-dimensional tomographic image having a narrower imaging range and a higher resolution than those of the three-dimensional tomographic image captured in the first imaging step. In this case, the three-dimensional tomographic image in the range that the user wants to check in detail is additionally captured with a high resolution, and thus a state of the tissue can be checked more appropriately. However, an additional imaging pattern may be set in advance.

In the additional imaging condition setting step, imaging conditions other than the above imaging pattern may be set together with the additional imaging pattern or instead of the additional imaging pattern. For example, at least one of a resolution of a two-dimensional tomographic image in a direction in which scanning with the measurement light is performed (referred to as “the number of A scan points” in some cases), the number of images to be added in a case where an addition averaging process is executed on a plurality of tomographic images captured at the same position, the OCT sensitivity, and the like may be set as additional imaging conditions. At least one of an optical path length of OCT, a focus condition, a polarization condition, a position of the internal fixation lamp, and the like at the time of additional imaging may be set as the additional imaging conditions. In this case, an image at a position that the user wants to check in detail is additionally captured under the set appropriate conditions.

At least a part of the range of the tomographic image additionally captured in the second imaging step may extend outside the imaging range of the three-dimensional tomographic image captured in the first imaging step. Also in this case, the user can appropriately ascertain an additional imaging position after checking the state of the tissue from the two-dimensional tomographic image extracted from the three-dimensional tomographic image. For example, in a case where a position and a range of additional imaging are set by setting the center of the range of the tomographic image to be additionally captured, the center of the range of additional imaging may be set within the imaging range of the three-dimensional tomographic image captured in the first imaging step.

The controller may further execute a front image display step and a position reception step. In the front image display step, the controller displays, on the display unit, a two-dimensional front image in a case where the tissue of which the three-dimensional tomographic image is captured is viewed from the direction along the optical axis of the measurement light. In the position reception step, the controller receives an instruction from the user for designating a position on the displayed two-dimensional front image. The controller may set at least one of an extraction position for extracting the two-dimensional tomographic image from the three-dimensional tomographic image and an additional imaging position in response to an instruction designated by the user. In this case, the user can ascertain the additional imaging position while checking the position when the tissue is viewed from the front on the two-dimensional front image and checking the internal state of the tissue from the tomographic image. Therefore, the additional capturing of the tomographic image is performed more appropriately.

The controller may further execute a position display step of displaying each of the extraction position of the two-dimensional tomographic image and the additional imaging position on the two-dimensional front image. In this case, the user can check both the extraction position of the two-dimensional tomographic image and the additional imaging position on the two-dimensional front image of the tissue. Therefore, the internal state of the tissue and the additional imaging position can be ascertained more appropriately.

A specific display method of the extraction position and the additional imaging position may be selected as appropriate. For example, in a case where a two-dimensional tomographic image is extracted from a three-dimensional tomographic image according to a set line pattern, the controller may display the set line pattern on the two-dimensional front image as an extraction position. The controller may display a line scanned with the measurement light at the time of the additional capturing as an additional imaging position on the two-dimensional front image. The controller may display an imaging region in which the additional capturing is performed on a two-dimensional front image.

In the position reception step, the controller may receive an instruction for collectively designating both the extraction position and the additional imaging position. In the position display step, the controller may move both the extraction position and the additional imaging position on the two-dimensional front image in conjunction with each other in response to an instruction input by the user. ln this case, since the extraction position and the additional imaging position are linked, the user can set the additional imaging position without any change while checking the internal state of the tissue at the extraction position from the two-dimensional tomographic image. Therefore, additional capturing is performed more appropriately.

The controller may receive an instruction from the user for designating a position in a state in which the two-dimensional front image that is a still image is displayed on the display unit. The controller may acquire a front observation image in real time when the tissue is viewed from the direction along the optical axis of the measurement light. On the basis of the two-dimensional front image and the front observation image, the controller may specify the additional imaging position that is designated on the two-dimensional front image on the front observation image captured in real time, and may perform additional capturing of the tomographic image at the specified additional imaging position. In this case, since the user can designate a position on the still image, the position is designated more easily and accurately than in a case where a position is designated on a moving image. After the additional imaging position designated on the still image is specified on the front observation image captured in real time, the additional capturing is performed. Therefore, even in a case where the tissue is moving, the additional capturing is accurately executed at the additional imaging position designated on the still image.

A method of specifying the additional imaging position designated on the still image on the front observation image captured in real time and automatically tracking the image may be selected as appropriate. For example, the controller may specify an additional imaging position on the front observation image by aligning the two-dimensional front image and the front observation image by using well-known image processing or the like. The controller may repeatedly perform the additional capturing while tracing (automatically tracking) the same position on the two-dimensional front image by using the front observation image captured in real time.

The controller may receive an instruction from the user for designating a position in a state in which an Enface image when the three-dimensional tomographic image is viewed from the direction along the optical axis of the measurement light is displayed on the display unit as a two-dimensional front image. In this case, an extraction position (which can be displayed as a line) of the two-dimensional tomographic image on the Enface image and a position where the two-dimensional tomographic image is actually extracted from the three-dimensional tomographic image completely match. Therefore, the user can ascertain an additional imaging position after checking an internal state of the tissue more accurately.

The controller may generate an analysis map that two-dimensionally represents a distribution of an analysis result of the three-dimensional tomographic image. The controller may receive an instruction from the riser for designating a position in a state in which the analysis map is displayed on the display unit as a two-dimensional front image. In this case, the user can designate at least one of the extraction position and the additional imaging position in consideration of the distribution of the analysis result. Therefore, the additional capturing is performed more appropriately. As in the case where a position is designated on the Enface image, an extraction position of the two-dimensional tomographic image does not deviate.

A specific aspect of the analysis map may be selected as appropriate. For example, the analysis map may be a thickness map that two-dimensionally represents a distribution of a thickness of a specific layer in the tissue. In this case, the user can designate a position after appropriately ascertaining a thickness of the specific layer.

The controller may further execute an image input step and a certainty factor information acquisition step. In the image input step, the controller inputs the three-dimensional tomographic image captured in the first imaging step to a mathematical model that is trained by using, a machine learning algorithm and executes analysis on at least one of a specific structure and a disease of the subject eye captured in an input ophthalmic image. In the certainty factor information acquisition step, the controller acquires certainty factor information indicating the certainty factor of the analysis executed on the three-dimensional tomographic image by the mathematical model. The controller may display the two-dimensional front image including the certainty factor information on the display unit.

In the mathematical model trained by using a plurality of ophthalmic images, the certainty factor of the analysis of the ophthalmic images tends to be higher when an ophthalmic image similar to the ophthalmic image used for training is input. On the other hand, when an ophthalmic image that is not similar to the ophthalmic image used for training is input to the mathematical model, the certainty factor of analysis of the ophthalmic image tends to be low. Therefore, when a three-dimensional tomographic image of a tissue in which some abnormality (for example, a disease) is present is input to the mathematical model, the certainty factor of a site where the abnormality is present tends to be low. Therefore, the user can easily check a state of a site where an abnormality is likely to be present by referring to the two-dimensional tomographic image including the certainty factor information.

A specific method of displaying a two-dimensional front image including certainty factor information may also be selected as appropriate. For example, the controller may display a certainty factor map two-dimensionally representing a distribution of certainty factor information as a two-dimensional front image or to be superimposed on the two-dimensional front image. The controller may add a certainty factor value or the like on the two-dimensional front image.

The controller may set at least one of the extraction position of the two-dimensional tomographic image and the additional imaging position on the basis of an analysis result of the three-dimensional tomographic image. In this case, a position is automatically determined on the basis of the analysis result. Therefore, the additional imaging position can be ascertained more appropriately.

A specific method of setting the position on the basis of the analysis result of the three-dimensional tomographic image may also be selected as appropriate. For example, the controller may perform image processing that is a kind of analysis process on the three-dimensional tomographic image to specify a feature site (for example, at least one of the optic disk, the macula, a blood vessel, and a lesion site) present in the imaging region. The controller may set a position of the specified feature site as at least one of the extraction position and the additional imaging position. The controller may automatically set at least one of the extraction position and the additional imaging position on the basis of the analysis result of the thickness of the specific layer in the tissue. As described above, the controller may execute the image input step and the certainty factor information acquisition step for acquiring the certainty factor information, and may automatically set at least one of the extraction position and the additional imaging position on the basis of the acquired certainty factor. For example, a position where the certainty factor is the lowest or a position where the certainty factor is equal to or less than a threshold value may be set to at least one of the extraction position and the additional imaging position.

The controller may store data regarding the three-dimensional tomographic image captured in the first imaging step and data regarding the additionally captured image captured in the second imaging step for the same tissue of the subject eye in the storage device in linking to each other. The controller may display simultaneously or in a switching manner between the two-dimensional tomographic image extracted from the three-dimensional tomographic image and the additionally captured image stored to be linked to the same three-dimensional tomographic image together with the data regarding the three-dimensional tomographic image (for example, at least one of the three-dimensional tomographic image and the analysis result of the three-dimensional tomographic image) on the display unit. In this case, in a state in which the data regarding the three-dimensional tomographic image is displayed, the two-dimensional tomographic image extracted from the three-dimensional tomographic image and the additionally captured image of the same tissue are displayed in a state in which the images can be easily compared. Therefore, the user's convenience is further improved.

A specific method for displaying the extracted two-dimensional tomographic image and the additionally captured image may be selected as appropriate. For example, the controller may display a graphical user interface (GUI; for example, an icon) for the user to input an instruction for switching display between the extracted two-dimensional tomographic image and the additionally captured image when displaying the data regarding the three-dimensional tomographic image on the display unit. The controller may switch the display between the two-dimensional tomographic image and the additionally captured image each time the GUI is operated.

### Embodiment

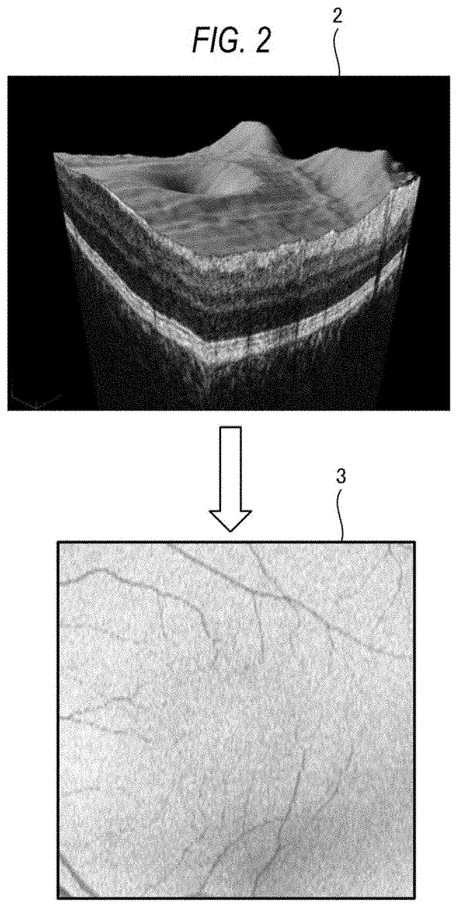

Hereinafter, one of the typical embodiments according to the present disclosure will be described. As an example, an OCT apparatus 1 of the present embodiment may use the fundus of a subject eye E as a subject and acquire and process OCT data of a fundus tissue (for example, a three-dimensional tomographic image 2 (refer to FIG. 2) and a two-dimensional tomographic image). However, at least some of the techniques exemplified in the present disclosure can also be applied in a case of processing OCT data regarding a tissue (for example, an anterior eye portion of the subject eye E) other than the fundus in the subject eye E or a subject (for example, a skin, a digestive organ, a brain, a blood vessel (including a heart blood vessel) or a tooth) other than the subject eye E. The OCT data is data acquired on the basis of the principle of optical coherence tomography (OCT).

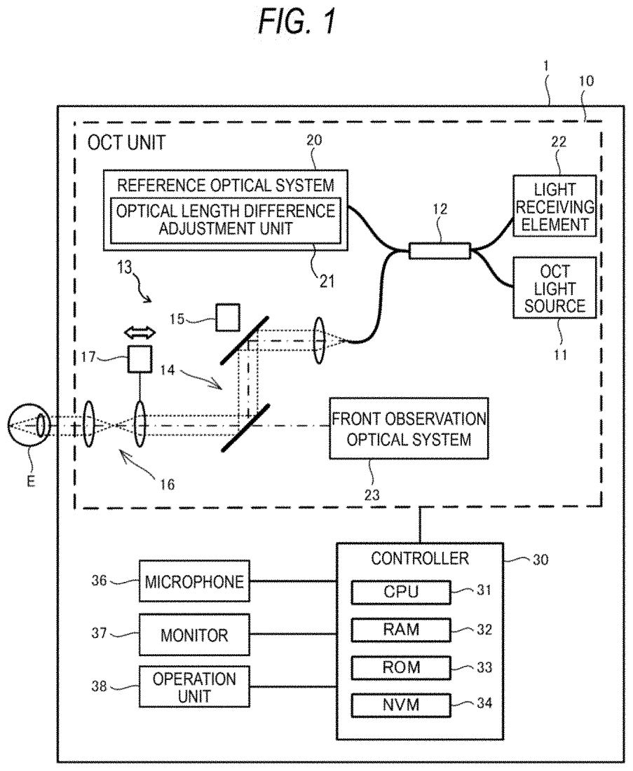

A schematic configuration of the OCT apparatus 1 of the present embodiment will be described with reference to FIG. 1. The OCT apparatus 1 includes an OCT unit 10 and a controller 30. The OCT unit 10 includes an OCT light source 11, a coupler (light splitter) 12, a measurement optical system 13, a reference optical system 20, a light receiving element 22, and a front observation optical system 23.

The OCT light source 11 emits light (OCT light) for acquiring OCT data. The coupler 12 divides the OCT light emitted from the OCT light source 11 into measurement light and reference light. The coupler 12 of the present embodiment combines the measurement light reflected by the subject (in the present embodiment, the fundus of the subject eye E) and the reference light generated by the reference optical system 20 to interfere with each other. That is, the coupler 12 of the present embodiment serves as a branch optical element that branches the OCT light into the measurement light and the reference light and a combined wave optical element that combines the reflected light of the measurement light and the reference light. A configuration of at least one of the branch optical element and the combined wave optical element may also be changed. For example, an element (for example, a circulator or a beam splitter) other than a coupler may be used.

The measurement optical system 13 guides the measurement light divided by the coupler 12 to the subject, and returns the measurement light reflected by the subject to the coupler 12. The measurement optical system 13 includes a scanning unit 14, an irradiation optical system 16, and a focus adjustment unit 17. By being driven by a drive unit 15, the scanning unit 14 can perform scanning with (deflect) the measurement light in a two-dimensional direction intersecting an optical axis of the measurement light. In the present embodiment, two galvanometer mirrors capable of deflecting the measurement light in different directions are used as the scanning unit 14. However, another device that deflects light (for example, at least one of a polygon mirror, a resonant scanner, and an acoustic optical element) may be used as the scanning unit 14. The irradiation optical system 16 is provided on the downstream side (that is, the subject side) of the optical path from the scanning unit 14, and irradiates the tissue of the subject with the measurement light. The focus adjustment unit 17 adjusts a focus of the measurement light by moving an optical member (for example, a lens) included in the irradiation optical system 16 in a direction along the optical axis of the measurement light.

The reference optical system 20 generates reference light and returns the reference light to the coupler 12. The reference optical system 20 of the present embodiment generates the reference light by reflecting the reference light divided by the coupler 12 by using a reflection optical system (for example, a reference mirror). However, a configuration of the reference optical system 20 may also be changed. For example, the reference optical system 20 may transmit the light incident from the coupler 12 without reflecting the incident light to be returned to the coupler 12. The reference optical system 20 includes an optical path length difference adjustment unit 21 that changes an optical path length difference between the measurement light and the reference light. In the present embodiment, the optical path length difference is changed by moving the reference mirror in the optical axis direction. A configuration for changing the optical path length difference may be provided in the optical path of the measurement optical system 13.

The light receiving element 22 detects an interference signal by receiving the interference light between the measurement light and the reference light generated by the coupler 12. In the present embodiment, the principle of Fourier domain OCT is employed. In the Fourier domain OCT, the spectral intensity (spectral interference signal) of the interference light is detected by the light receiving element 22, and a complex OCT signal is acquired by performing Fourier transform on the spectral intensity data. As an example of the Fourier domain OCT, any of spectral-domain-OCT (SD-OCT), swept-source-OCT (SS-OCT), and the like may be employed. For example, time-domain-OCT (TD-OCT) may be employed.

In the present embodiment, the SD-OCT is employed. In the case of the SD-OCT, for example, a low coherent light source (broadband light source) is used as the OCT light source 11, and spectroscopic optical system (spectrometer) that disperses the interference light into each frequency component (each wavelength component) is provided in the vicinity of the light receiving element 22 in the optical path of the interference light. In the case of the SS-OCT, as the OCT light source 11, for example, a wavelength scanning type light source (wavelength variable light source) that temporally changes an emission wavelength at a high speed is used. In this case, the OCT light source 11 may include a light source, a fiber ring resonator, and a wavelength selection filter. Examples of the wavelength selection filter include, for example, a filter that combines a diffraction grating and a polygon mirror, and a filter that uses the Fabry-Perot etalon.

In the present embodiment, scanning with a spot of the measurement light is performed by the scanning unit 14 in a two-dimensional measurement region, and thus three-dimensional OCT data (three-dimensional tomographic image) is acquired. However, the principle of acquiring three-dimensional OCT data may also be changed. For example, three-dimensional OCT data may be acquired on the basis of the principle of line field OCT (hereinafter referred to as “LF-OCT”). In the LF-OCT, the measurement light is simultaneously applied on an irradiation line extending in the one-dimensional direction in the tissue, and the interference light between the reflected light of the measurement light and the reference light is received by a one-dimensional light receiving element (for example, a line sensor) or a two-dimensional light receiving element. In the two-dimensional measurement region, scanning with the measurement light is performed in a direction intersecting the irradiation line, and thus the three-dimensional OCT data is acquired. The three-dimensional OCT data may be acquired on the basis of the principle of full-field OCT (hereinafter, referred to as “FF-OCT”). In the FF-OCT, the measurement light is applied to the two-dimensional measurement region on the tissue, and the interference light between the reflected light of the measurement light and the reference light is received by a two-dimensional light receiving element. In this case, the OCT apparatus 1 does not have to include the scanning unit 14.

The front observation optical system 23 is provided for capturing a front observation image of the tissue of the subject (in the present embodiment, the fundus of the subject eye E) in real time. The front observation image in the present embodiment is a two-dimensional image when the tissue is viewed from the direction (front direction) along the optical axis of the measurement light of the OCT. In the present embodiment, a scanning laser ophthalmoscope (SLO) is employed as the front observation optical system 23. However, for the configuration of the front observation optical system 23, a configuration (for example, an infrared camera that collectively irradiates a two-dimensional imaging range with infrared light to capture a front image) other than an SLO may be employed.

As shown in FIG. 2, the OCT apparatus 1 can acquire (generate) an Enface image 3 that is a two-dimensional front image when the tissue is viewed from the direction (front direction) along the optical axis of the measurement light on the basis of the captured three-dimensional tomographic image 2. In a case where the Enface image 3 is acquired in real time, the acquired Enface image 3 may also be used as the above front observation image. In this case, the front observation optical system 23 may be omitted. Data regarding the Enface image 3 may be, for example, integrated image data in which luminance values are integrated in a depth direction (Z direction) at respective positions in the XY direction, integrated values of spectral data at respective positions in the XY direction, and luminance data at each position in the XY direction in a certain depth direction, or luminance data at each position in the XY direction in any layer of the retina (for example, the surface layer of the retina). The Enface image 3 may be obtained from a motion contrast image (for example, an OCT angiography image) obtained by acquiring a plurality of OCT signals from the same position in the tissue of the patient's eye at different times.

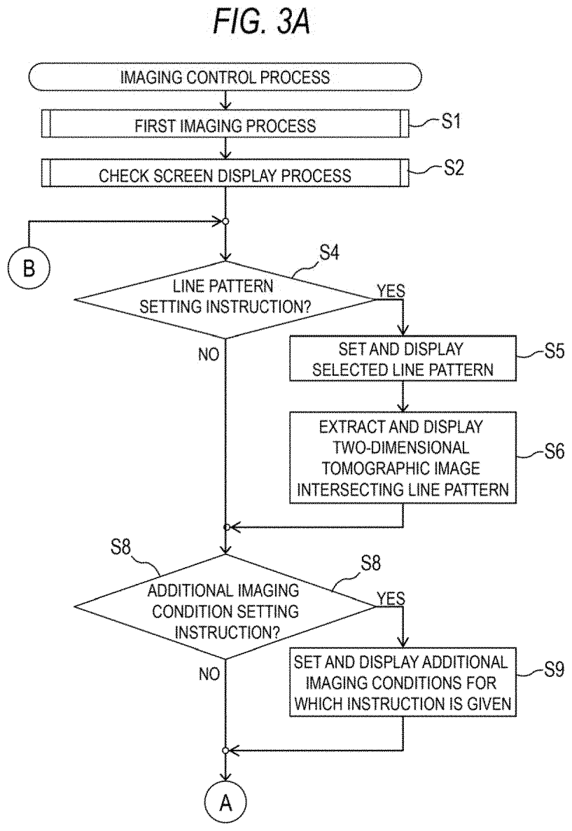

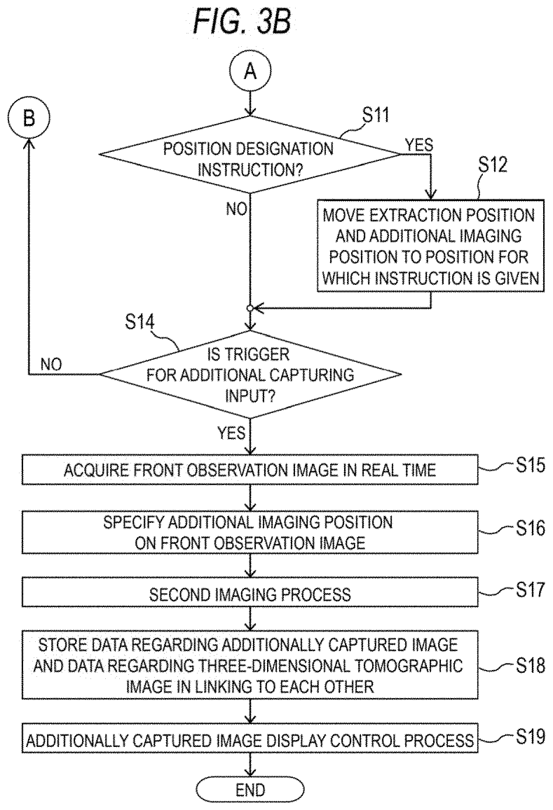

The controller 30 performs various types of control of the OCT apparatus 1. The controller 30 includes a CPU 31, a RAM 32, a ROM 33, and a nonvolatile memory (NVM) 34. The CPU 31 is a controller that performs various types of control. The RAM 32 temporarily stores various types of information. The ROM 33 stores a program executed by the CPU 31, various initial values, and the like. The NVM 34 is a non-transitory storage medium capable of storing storage contents even when the power supply is cut off. An imaging control program for executing an imaging control process (refer to FIGS. 3A and 3B) that will be described later may be stored in the NVM 34.

A microphone 36, a monitor 37, and an operation unit 38 are connected to the controller 30. The microphone 36 receives sound. The monitor 37 is an example of a display unit that displays various images. The operation unit 38 is operated by a user in order for the user to input various operation instructions to the OCT apparatus 1. For the operation unit 38, for example, various devices such as a mouse, a keyboard, a touch panel, and a foot switch may be used. Various operation instructions may be input to the OCT apparatus 1 by inputting sound to the microphone 36. In this case, the CPU 31 may determine the type of operation instruction by performing a voice recognition process on the input sound.

In the present embodiment, the integrated OCT apparatus 1 in which the OCT unit 10 and the controller 30 are built in one casing is exemplified. However, needless to say, the OCT apparatus 1 may include a plurality of devices having different casings. For example, the OCT apparatus 1 may include an optical device in which the OCT unit 10 is built and a PC connected to the optical device by wire or wirelessly. In this case, a controller included in the optical device and a controller of the PC may both function as the controller 30 of the OCT apparatus 1. A device located at a base where an image of the subject eye is captured and a device located at a base of the user (for example, a doctor) may be connected via a network. In this case, even if the user is at a base different from the imaging base, the user can give an instruction for the additional capturing at an appropriate position through a process described below.

### Imaging Control Process

The imaging control process executed by the OCT apparatus 1 will be described with reference to FIGS. 3A to 10. In the imaging control process of the present embodiment, first, the three-dimensional tomographic image 2 of the tissue of the subject eye is captured. By using the captured three-dimensional tomographic image 2, various processes for appropriately performing additional capturing of a tomographic image are executed. The CPU 31 of the OCT apparatus 1 executes the imaging control process shown in FIGS. 3A and 3B according to the imaging control program stored in the NVM 34. First, the CPU 31 executes a first imaging process (S1). In the first imaging process, the three-dimensional tomographic image 2 is captured.

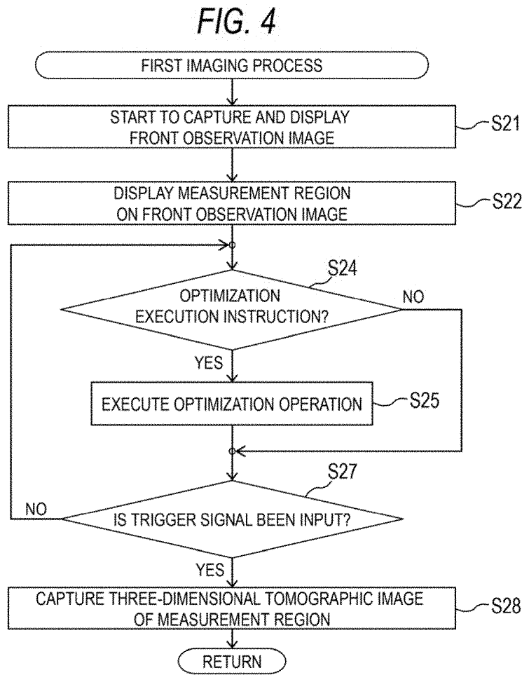

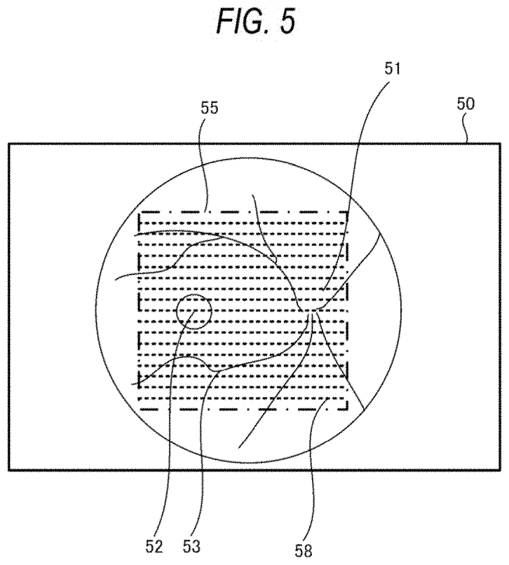

The first imaging process will be described in detail with reference to FIG. 4. The CPU 31 starts capturing a front observation image of the tissue that is an image capturing target (in the present embodiment, the fundus of the subject eye E), and displays the captured image on the monitor 37 (S21). FIG. 5 shows an example of a front observation image 50 displayed on the monitor 37. In the example shown in FIG. 5, an optic disk (hereinafter, also referred to as a “papilla”) 51, the macula 52, and a fundus blood vessel 53 of the subject eye E are captured in the front observation image 50. The front observation image 50 is repeatedly and intermittently captured (that is, captured in real time) and displayed as a moving image on the monitor 37. As described above, the front observation image 50 in the present embodiment is an image (an SLO image in the present embodiment) captured by the front observation optical system 23 (refer to FIG. 1). However, as described above, the Enface image 3 or an infrared image may be used as the front observation image 50.

Next, the CPU 31 displays a measurement region 55 on the front observation image 50 (S22). The measurement region 55 is a region of the tissue that is a target of which the three-dimensional tomographic image 2 is captured. The measurement region 55 is a two-dimensional region expanding in a direction intersecting the optical axis of the measurement light. When an operation of capturing the three-dimensional tomographic image 2 is started, the measurement light is applied into the measurement region 55.

In the example shown in FIG. 5, a frame portion of the measurement region 55 is electronically displayed on the front observation image 50. However, a display method of the measurement region 55 may be changed as appropriate. For example, the tissue may be directly irradiated with light indicating the frame portion of the measurement region 55. In this case, the user can ascertain the measurement region 55 by checking the position of the light reflected in the front observation image 50. In the example shown in FIG. 5, the measurement region 55 is rectangular. However, needless to say, a shape of the measurement region 55 may be a shape other than a rectangular shape (for example, a circular shape).

While checking the front observation image 50, the user performs alignment of the OCT apparatus 1 with respect to the subject and adjusts the measurement region 55 to an appropriate position with respect to the tissue. In the present embodiment, in order to cause both the papilla 51 and the macula 52 to be included in the measurement region 55, the center of the measurement region 55 is adjusted to be located between the papilla 51 and the macula 52. The alignment of the OCT apparatus 1 with respect to the subject may be automatically performed.

Next, the CPU 31 determines whether or not an instruction for executing optimization has been received (S24). The optimization is a function of adjusting an optical path length difference by the optical path length difference adjustment unit 21 (refer to FIG. 1) and optimizing a focus by the focus adjustment unit 17. When the optimization execution instruction is received (S24: YES), the CPU 31 executes an optimization operation (S25), and the process proceeds to S7. If the optimization execution instruction is not received (S24: NO), the process proceeds to S27 without further processing.

Next, the CPU 31 determines whether or not a trigger signal for starting to capture the three-dimensional tomographic image 2 has been generated (has been input in the present embodiment) (S27). As an example, in the present embodiment, when the user operates a release button (not shown) for giving an instruction for the start of imaging in a state in which the alignment and the optimization operation are completed, a trigger signal is generated and input to the CPU 31. However, a method of generating a trigger signal may be changed. For example, a trigger signal may be generated by inputting a specific voice to the microphone 36. The CPU 31 may automatically generate a trigger signal when the imaging preparation (for example, alignment and optimization operation) is completed. If the trigger signal is not generated (S27: NO), the process returns to S24.

When the trigger signal is generated (S27: YES), the CPU 31 executes a process for capturing the three-dimensional tomographic image 2 of the measurement region 55 of the tissue (S28). The CPU 31 of the present embodiment controls the scanning unit 14 to scan the two-dimensional measurement region 55 with the spot of the measurement light, and thus the three-dimensional tomographic image 2 of the measurement region 55 is captured. As an example, in the present embodiment, as shown in FIG. 5, a plurality of linear scanning lines (scan lines) 58 for performing scanning with spots are set in the measurement region 55 at equal intervals, and scanning with the spot of the measurement light is performed on each scanning line 58 such that the three-dimensional tomographic image 2 of the measurement region 55 is captured.

The OCT apparatus 1 of the present embodiment can capture a tomographic image while tracking a scanning position of the spot of the measurement light by using a two-dimensional front image (template image) of a reference tissue and a front observation image captured in real time by the front observation optical system 23. By performing tracking at the time of imaging, a tomographic image at an accurate position is captured even in a case where the eyes move. The user may also set in advance whether or not to execute tracking at the time of imaging.

The OCT apparatus 1 of the present embodiment can capture a plurality of tomographic images at the same position by performing scanning with the measurement light a plurality of times on the same scanning line 58. The OCT apparatus 1 may perform an addition averaging process on a plurality of tomographic images captured at the same position. The image quality of the tomographic image is improved by performing the addition averaging process. The user may also set in advance whether or not to execute the addition averaging process.

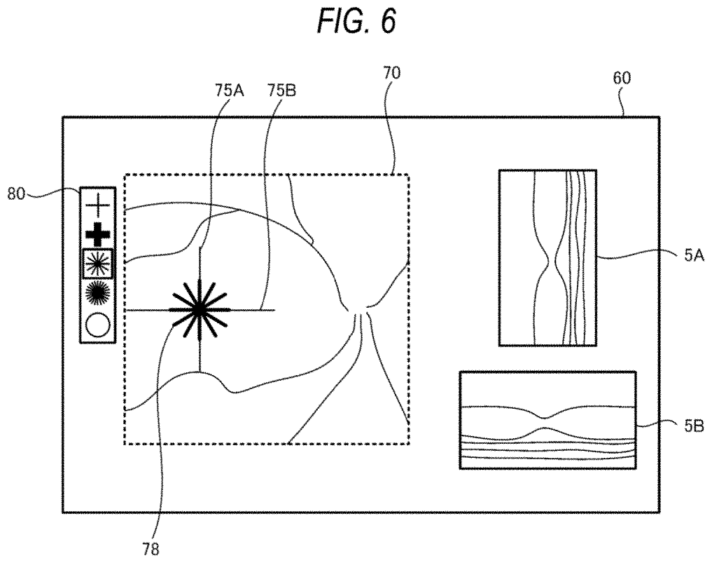

FIGS. 3A and 3B will be referred to again. When the first imaging process (S1) is finished, various processes for optimizing additional capturing of a tomographic image are executed. First, the CPU 31 executes a check screen display process for displaying an imaging result check screen 60 (refer to FIG. 6) on the monitor 37 (S2).

The imaging result check screen 60 in the present embodiment will be described with reference to FIG. 6. The imaging result check screen 60 is displayed such that the user can check the image captured in the first imaging process (refer to FIG. 4). The imaging result check screen 60 of the present embodiment is displayed in order to optimize additional capturing of a tomographic image. As shown in FIG. 6, a two-dimensional front image 70, the two-dimensional tomographic images 5 (5A and 5B), and an additional imaging pattern selection unit 80 are displayed on the imaging result check screen 60 of the present embodiment.

The two-dimensional front image 70 is a two-dimensional image when the tissue of which the three-dimensional tomographic image 2 is captured is viewed from the direction (that is, the front) along the optical axis of the measurement light of the OCT. The OCT apparatus 1 of the present embodiment allows the user to check an extraction position of the two-dimensional tomographic image 5 extracted and displayed from the three-dimensional tomographic image 2, an additional imaging position of a tomographic image, and the like on the two-dimensional front image 70.

As an example, the OCT apparatus 1 of the present embodiment allows the user to check the extraction position of the two-dimensional tomographic image 5 by displaying a line pattern 75 (75A and 75B) on the two-dimensional front image 70. In other words, the OCT apparatus 1 sets a line pattern for the two-dimensional measurement region 55 (overlapping the two-dimensional front image 70) in which the three-dimensional tomographic image 2 is captured, and a two-dimensional tomographic image 5 in a line of the set line pattern (for example, the two-dimensional tomographic image 5 in a section intersecting the line) is displayed on the imaging result check screen 60. In the example shown in FIG. 6, a line pattern in which a line 75A extending vertically and a line 75B extending horizontally are crossed is set. The OCT apparatus 1 extracts the two-dimensional tomographic image 5A in the line 75A and the two-dimensional tomographic image 5B in the line 75B from the three-dimensional tomographic image 2 and displays the images on the monitor 37.

The OCT apparatus 1 of the present embodiment displays an additional imaging pattern 78 on the two-dimensional front image 70 such that the user checks an additional imaging position of a tomographic image (to be exact, additional imaging candidate positions in the state shown in FIG. 6). In other words, the OCT apparatus 1 sets and displays the selected additional imaging pattern 78 at the additional imaging position on the two-dimensional front image 70 in response to an instruction from the user or the like. When an instruction for executing the additional capturing is received, the OCT apparatus 1 executes additional capturing of a tomographic image by performing scanning with the measurement light on the line of the additional imaging pattern 78 set at the time of receiving the instruction.

As will be described later in detail, the OCT apparatus 1 of the present embodiment receives an instruction from the user for designating a position (in the present embodiment, an extraction position of the two-dimensional tomographic image 5 and an additional imaging position) on the two-dimensional front image 70 that is a still image. In this case, since the user can designate a position on the still image, the position is designated more easily and accurately than in a case where a position is designated on a moving image. However the OCT apparatus 1 may also receive an instruction from the user for designating a position on a moving image.

The additional imaging pattern selection unit 80 is displayed to allow the user to select at least one of a plurality of types of additional imaging patterns. As an example, in the additional imaging pattern selection unit 80 shown in FIG. 6, additional imaging patterns such as cross, multi (cross), radial (6), radial (12), and circle are displayed as candidates in order from the top. In the example shown in FIG. 6, as a result of the user selecting the radial (6), the radial (6) in the additional imaging pattern selection unit 80 is highlighted, and the additional imaging pattern 78 of the radial (6) is displayed at the additional imaging position on the two-dimensional front image 70. The additional imaging patterns may include a pattern for capturing a three-dimensional tomographic image having a narrower imaging range and a higher resolution than those of the three-dimensional tomographic image 2 captured in the first imaging process (refer to FIG. 4).

The OCT apparatus 1 of the present embodiment overlaps a region for setting an extraction position (positions of the line pattern 75) of the two-dimensional tomographic image 5 and a region for setting the additional imaging position (a position of the additional imaging pattern 78). Specifically, the OCT apparatus 1 of the present embodiment sets the extraction position and the additional imaging position in a state in which the center of the extraction position (a position of the line pattern 75) of the two-dimensional tomographic image 5 and the center of the additional imagine position (a position of the additional imaging pattern 78) match each other. Therefore, the user can start the additional capturing after appropriately ascertaining an internal state of the tissue at the additional imaging position from the extracted and displayed two-dimensional tomographic image 5. However, the extraction position of the two-dimensional tomographic image 5 and the additional imaging position of the tomographic image may be set separately.

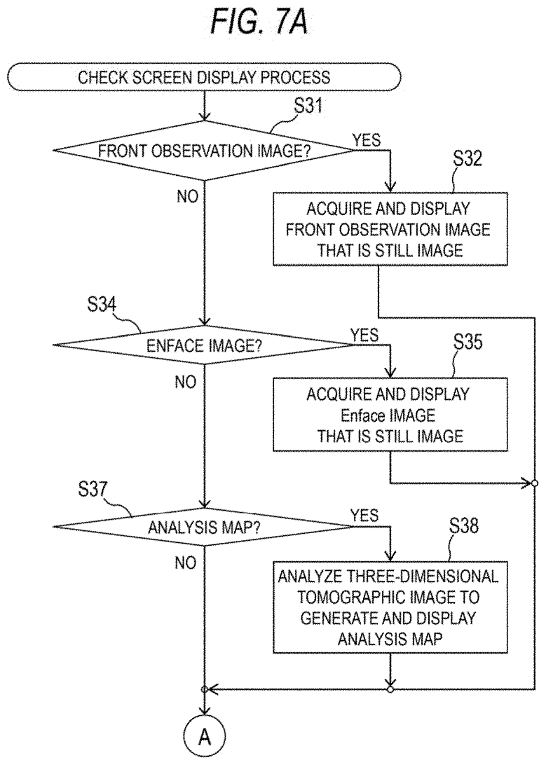

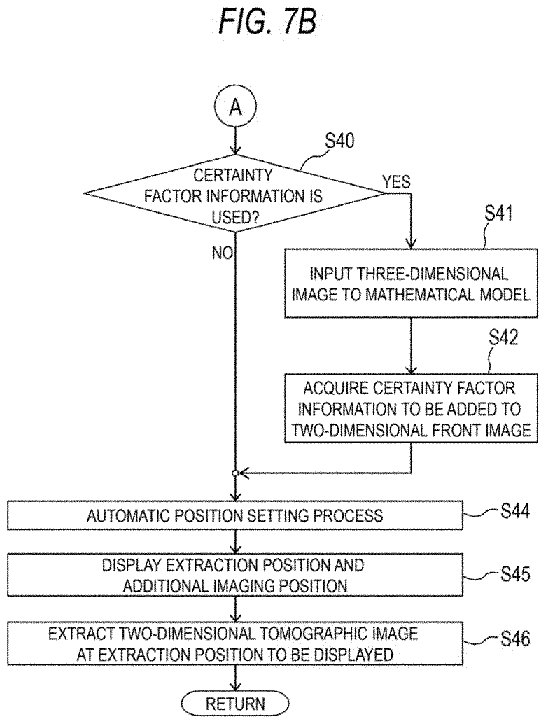

The check screen display process (refer to S2 in FIG. 3A) will be described in detail with reference to FIGS. 7A and 7B. First, the CPU 31 executes a process for displaying the two-dimensional front image 70 on the imaging result check screen 60 (S31 to S42). As an example, in the present embodiment, as the two-dimensional front image 70, any of a front observation image captured by the front observation optical system 23, the Enface image 3 (refer to FIG. 2), the analysis map 73, and a certainty factor display image 74 may be displayed on the monitor 37. The user can set in advance the type of an image to be displayed as the two-dimensional front image 70 among a plurality of types of images on a setting screen or the like.

In a case where the front observation image is to be displayed as the two-dimensional front image 70 (S31: YES), the CPU 31 acquires a front observation image that is a still image captured by the front observation optical system 23, and displays the front observation image on the monitor 37 (S32). Thereafter, the process proceeds to S40. The front observation image displayed in S32 is preferably all image captured by the front observation optical system 23 when the three-dimensional tomographic image 2 is captured.

In a case where the Enface image 3 is set to be displayed as the two-dimensional front image 70 (S34: YES), the CPU 31 acquires the Enface image 3 (refer to FIG. 2) that is a still image and displays the Enface image 3 on the monitor 37 (S35). Thereafter, the process proceeds to S40. The Enface image 3 displayed in S35 is acquired (generated) on the basis of the three-dimensional tomographic image 2 captured in the first imaging step (refer to FIG. 4). Therefore, an extraction position of the two-dimensional tomographic image on the Enface image 3 (for example, the position of the line pattern 75 shown in FIG. 6) and a position where the two-dimensional tomographic image 5 is actually extracted from the three-dimensional tomographic image 2 completely match each other. Therefore, an internal state of the tissue can be checked more accurately.

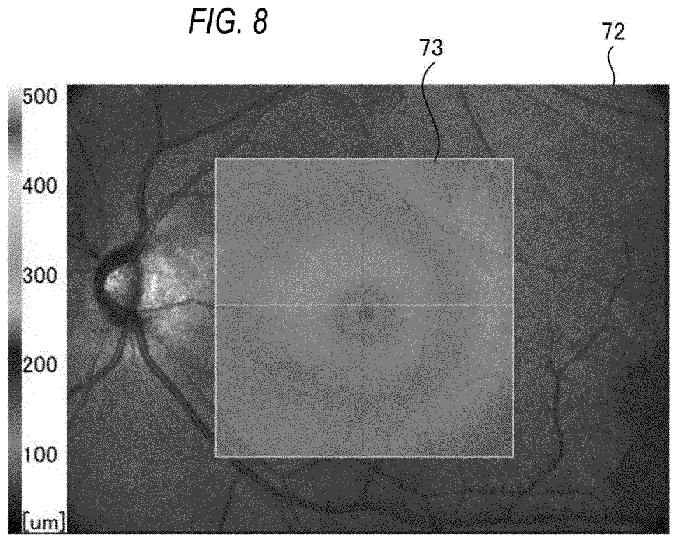

In a case where the analysis map 73 is set to be displayed as the two-dimensional front image 70 (S37: YES), the CPU 31 analyzes the three-dimensional tomographic image 2 captured in the first imaging step (refer to FIG. 4), and generates an analysis map 73 that two-dimensionally represents a distribution of an analysis result. The CPU 31 displays the generated analysis map 73 on the monitor 37 (S38). Thereafter, the process proceeds to S40. The analysis map 73 of the present embodiment is a still image. In the example shown in FIG. 8, the analysis map 73 is superimposed and displayed on a two-dimensional region where the analysis is executed in the image region of the front observation image 72 captured by the front observation optical system 23. Therefore, the user can ascertain the analysis result from the analysis map 73 while checking a position of the tissue or the like from the front observation image 72. However, a display method of the analysis map 73 may also be changed. For example, the analysis map 73 may be independently displayed as the two-dimensional front image 70.

The analysis map 73 exemplified in the present embodiment is a thickness map that two-dimensionally represents a distribution of a thickness of a specific layer (for example, a layer between ILM and RPE/BM) in the tissue with the change of colors. However, an analysis map (for example, a map representing a distribution of an analysis result regarding a blood flow in the fundus) different from the thickness map may be used. A map (so-called “deviation map”) representing a distribution of a difference between analysis result data for a normal eye (normal eye data) and an analysis result of the subject eye may be used.

Next, the CPU 31 determines whether or not the two-dimensional front image 70 is set to include certainty factor information (S40). The certainty factor information is information regarding a certainty factor of analysis executed by a mathematical model trained by using a machine learning algorithm. The “certainty factor” may be a high certainty of analysis of an ophthalmic image by the mathematical model, or may be a reciprocal of a low certainty (which can also be expressed as uncertainty). For example, in a case where the uncertainty is expressed by x%, the certainty factor may be a value expressed by (100-x)%. That is, the result is the same not only in a case where a value of a “certainty factor” indicating a high certainty of the analysis is used, but also in a case where a low certainty (uncertainty) of the analysis is used. In the mathematical model trained by using a plurality of ophthalmic images, the certainty factor of the analysis of the ophthalmic images tends to be higher when an ophthalmic image similar to the ophthalmic image used for training is input. On the other hand, when an ophthalmic image that is not similar to the ophthalmic image used for training is input to the mathematical model, the certainty factor of analysis of the ophthalmic image tends to be low. Therefore, when a three-dimensional tomographic image of a tissue in which some abnormality (for example, a disease) is present is input to the mathematical model, the certainty factor of a site where the abnormality is present tends to be low.

In a case where a two-dimensional front image including the certainty factor information is set to be used (S40: YES), the CPU 31 inputs the three-dimensional tomographic image 2 captured in the first imaging step (refer to FIG. 4) to the mathematical model trained by using the machine learning algorithm (S41). The mathematical model is trained to output an analysis result for at least one of specific structures and diseases of the subject eye captured in an input ophthalmic image. The certainty factor information is obtained accompanying an analysis result. The CPU 31 acquires certainty factor information regarding the analysis executed by the mathematical model and adds the certainty factor information onto the two-dimensional front image 70 (S42).

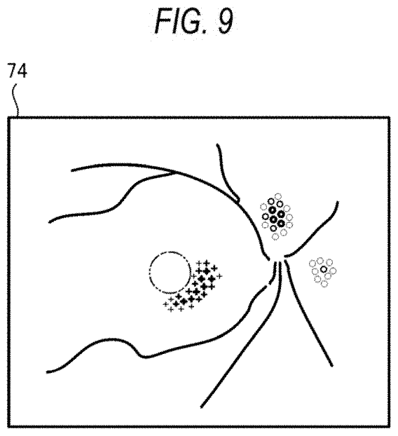

A specific method for adding the certainty factor information onto the two-dimensional front image 70 may be selected as appropriate. For example, in a certainty factor display image 74 shown in FIG. 9, the certainty factor information is added on an image of another two-dimensional front image 70 (for example, the front observation image 72 captured by the front observation optical system 23). Specifically, an indication indicating the degree of low certainty factor is added to a region of the two-dimensional front image 70 where the acquired certainty factor is less than a threshold value. Therefore, the user can appropriately ascertain a region where there is a high possibility that some abnormality may be present in the tissue. The certainty factor map representing a two-dimensional distribution of the certainty factor may be used as the two-dimensional front image 70.

Next, the CPU 31 executes a process of automatically setting an extraction position of the two-dimensional tomographic image 5 and an additional imaging position (S44). For example, if the automatically set additional imaging position is appropriate, the user may directly input an instruction for executing the additional capturing without further processing. Therefore, the user's work efficiency is improved.

As an example, in the present embodiment, the CPU 31 automatically sets an extraction position and an additional imaging position on the basis of an analysis result for the three-dimensional tomographic image 2. For example, the CPU 31 may execute image processing that is a kind of analysis process on the three-dimensional tomographic image 2 to specify a feature site (for example, at least one of the optic disk, the macula, a blood vessel, and a lesion site) present in the imaging region. The CPU 31 may set a position of the specified feature site as an extraction position and an additional imaging position. The CPU 31 may automatically set the extraction position and the additional imaging position on the basis of the analysis result of the thickness of the specific layer in the tissue. The CPU 31 may automatically set the extraction position and the additional imaging position on the basis of the above certainty factor information. For example, a position where the certainty factor is the lowest, a position where the certainty factor is equal to or less than a threshold value, or the like may be set as the extraction position and the additional imaging position. The CPU 31 may set a predefined default position (for example, the center of the two-dimensional front image 70) as at least one of the extraction position and the additional imaging position without using an analysis result of the three-dimensional tomographic image 2.

Next, the CPU 31 displays the extraction position of the two-dimensional tomographic image 5 and the additional imaging position on the two-dimensional front image 70 (S45). As described above, in the present embodiment, the set line pattern 75 among the plurality of types of line patterns is displayed at the extraction position. The set additional imaging pattern 78 among the plurality of types of additional imaging patterns is displayed at the additional imaging position. In S45, a pattern display process may be performed on the basis of the line pattern 75 and the additional imaging pattern 78 defined by default, a pattern set in the previous imaging control process, or the like. In the present embodiment, the extraction position and the additional imaging position overlap each other. Therefore, in the example shown in FIG. 6, the line pattern 75 and the additional imaging pattern 78 are displayed in an overlapping state.

Next, the CPU 31 extracts the two-dimensional tomographic image 5 at the set extraction position from the three-dimensional tomographic image 2 captured in the first imaging step (refer to FIG. 4) and displays the two-dimensional tomographic image 5 on the monitor 37 (S46). Specifically, in the present embodiment, the two-dimensional tomographic image 5 in the line of the set line pattern 75 is extracted from the three-dimensional tomographic image 2 and displayed on the monitor 37.

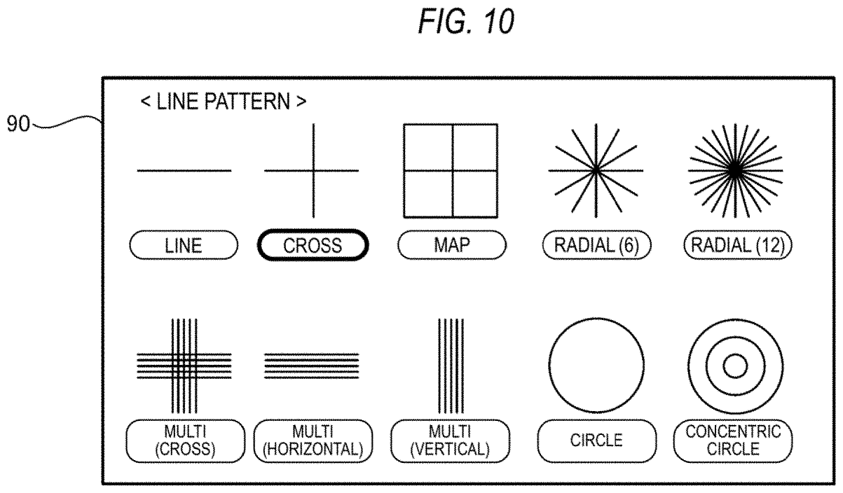

FIGS. 3A and 3B will be referred to again. When the check screen display process (S2) is finished, the CPU 31 determines whether or not a line pattern setting instruction for extracting the two-dimensional tomographic image 5 has been input (S4). In the present embodiment, the user can input an instruction for setting a line pattern by operating the operation unit 38. As an example, in the present embodiment, when a predetermined operation is performed on the operation unit 38, a line pattern setting screen shown in FIG. 10 is popped up and displayed on the monitor 37. The user can input a line pattern setting instruction to the OCT apparatus 1 by selecting a desired line pattern from among a plurality of types of line patterns displayed on a line pattern setting screen 90. Since a cross line pattern is set in the example shown in FIG. 10, the cross line pattern 75 is displayed on the imaging result check screen 60 shown in FIG. 6.

When the line pattern setting instruction is input (S4: YES), the CPU 31 sets the line pattern 75 selected by the user from among the plurality of types of line patterns and displays the line pattern 75 on the monitor 37 (S5). The CPU 31 extracts the two-dimensional tomographic image 5 in the line of the set line pattern 75 and displays the two-dimensional tomographic image 5 on the monitor 37. Thereafter, the process proceeds to S8.

Next, the CPU 31 determines whether or not an instruction for setting additional imaging conditions has been input (S8). In the present embodiment, the additional imaging conditions that can be set in S8 include an additional imaging pattern that is an additional imaging pattern of a tomographic image. As described above, the user can input an instruction for setting an additional imaging pattern by selecting a desired additional imaging pattern from among the plurality of types of additional imaging patterns displayed on the additional imaging pattern selection unit 80.

In S8 of the present embodiment, in addition to the additional imaging pattern, at least one of a resolution of a two-dimensional tomographic image in a direction in which scanning with the measurement light is performed (referred to as “the number of A scan points” in some cases), the number of images to be added in a case where an addition averaging process is executed, an OCT sensitivity, an optical path length of OCT, a focus condition (for example, a position of a focus in the depth direction), a polarization condition, and a position of an internal fixation lamp is set as the additional imaging conditions. For example, in S8, the user may also select a retinal mode and a choridal mode as a condition for the optical path length of the OCT. The retinal mode is a mode in which a zero delay position of an optical path length (a corresponding vertical position on the subject when an optical path length of the reference light and an optical path length of the measurement light match) is set at a position shallower than the surface layer of the retina. The choridal mode is a mode in which the zero delay position of the optical path length is set at a position deeper than the choroid layer. The user can more appropriately check a state of the tissue by selecting the retinal mode or the choridal mode according to a position to be checked in detail among respective positions in the depth direction. In the OCT apparatus 1, as a passing position of the measurement light approaches from the center of an objective lens to the periphery, an optical path becomes longer and the measurement light is more likely to be scattered, and eclipse of mechanical parts is more likely to occur, and thus the sensitivity is more likely to be lower. Therefore, a position of the internal fixation lamp may be set such that a scan position of the measurement light at the time of additional imaging is close to the center of the objective lens. As a result, the quality of an additionally captured image is improved.

When the instruction for setting additional imaging conditions is input (S8: YES), the CPU 31 sets an additional imaging condition for which an instruction is given by the user and displays the additional imaging condition 78 on the monitor 37. Thereafter, the process proceeds to S11.

Next, the CPU 31 determines whether or not an instruction for designating the extraction position and the additional imaging position has been input (S11). The user may input an instruction for designating an extraction position and an additional imaging position (for example, an instruction for moving the position) to the OCT apparatus 1 via the operation unit 38 or the like. For example, the CPU 31 may recognize a position of a pointer moving on the two-dimensional front image 70 in response to an operation instruction from the user as a position designated by the user. A position on the two-dimensional front image 70 may be designated by using a touch panel.

When the extraction position and the additional imaging position are designated (S11: YES), the CPU 31 moves the extraction position and the additional imaging position (in the present embodiment, the position of the line pattern 75 and the position of the additional imaging pattern 78) on the two-dimensional front image 70 displayed on the monitor 37 to the designated position (S12). That is, in the present embodiment, the CPU 31 moves both the extraction position and the additional imaging position on the two-dimensional front image 70 in conjunction with each other. Therefore, the user can set the additional imaging position without further processing while checking an internal state of the tissue at the extraction position from the two-dimensional tomographic image 5. However, the extraction position and the additional imaging position may be designated (moved) separately.

Next, the CPU 31 determines whether or not a trigger for executing the additional capturing of the tomographic image has been input (S14). In the present embodiment, a trigger for the additional capturing is input by various methods. For example, the user may also input an instruction for executing the additional capturing as a trigger after setting the additional imaging position on the two-dimensional front image 70. The CPU 31 may set an operation of setting the additional imaging position on the two-dimensional float image 70 as an operation of a trigger for the additional capturing. In this case, the user can cause the OCT apparatus 1 to execute the additional capturing only by setting the additional imaging position. The CPU 31 may compare the front observation image captured in real time with the two-dimensional front image 70 after the additional imaging position is set, and set matching between the additional imaging position set on the two-dimensional front image 70 and the additional imaging position on the front observation image as a trigger.

If the trigger for the additional capturing is not input (S14: NO), the process returns to S4, and the processes in S4 to S14 are repeatedly performed. When the trigger for the additional capturing is input (S14: YES), the CPU 31 acquires a front observation image captured in real time by the front observation optical system 23 (S15). On the basis, of the two-dimensional front image 70 and the front observation image, the CPU 31 specifies the additional imaging position designated on the two-dimensional front image 70 that is a still image, on the front observation image captured in real time (S16). A method of specifying the additional imaging position designated on the two-dimensional front image 70 on the front observation image may be selected as appropriate. In the present embodiment, the CPU 31 specifies the additional imaging position on the front observation image by aligning the two-dimensional front image 70 with the front observation image by using well-known image processing or the like.

The CPU 31 executes the second imaging process (S17). In the second imaging process, the CPU 31 executes the additional capturing of the tomographic image at the additional imaging position specified in S16 according to the additional imaging pattern 78 set in S9 (S17). Specifically, the CPU 31 executes additional capturing of a tomographic image by performing scanning with the measurement light on the line of the additional imaging pattern 78 selected by the user and set at the additional imaging position. According to the above process, the user can check an internal state of the tissue within the imaging range of the three-dimensional tomographic image 2 from the extracted two-dimensional tomographic image 5, and then cause the OCT apparatus 1 to execute the additional capturing at an appropriate position. The image quality of the additionally captured tomographic image is unlikely to deteriorate compared with the image quality of an image arbitrarily extracted from the three-dimensional tomographic image 2. Therefore, in a range in which the three-dimensional tomographic image 2 is captured, an image at a position that the user wants to check in detail is presented to the user more appropriately with higher image quality.

In the second imaging process (S17), the OCT apparatus 1 also executes tracking using the template image and the real-time front observation image in the same manner as in the first imaging process (refer to FIG. 4) described above. Here, as a template image used as a reference for tracking, for example, a front observation image (an SLO image in the present embodiment) captured at the start of tracking, a front observation image captured at a timing of imaging trigger input (release), or the Enface image 3 (refer to FIG. 2) generated on the basis of the three-dimensional tomographic image 2 may be used.

Here, which image is used as a template image as a reference for tracking may be automatically set on the basis of the imaging conditions when the three-dimensional tomographic image 2 is captured in the first imaging process. For example, in a case where the addition averaging process is executed in the first imaging process, it is unlikely that the Enface image 3 generated on the basis of the three-dimensional tomographic image 2 will be distorted and damaged, and thus the Enface image 3 may be used as a template image. On the other hand, in a case where the addition averaging process is not executed in the first imaging process, distortion and damage may occur in the Enface image 3, and thus a front observation image captured at the start of tracking or a front observation image captured at a release timing may be used as a template image.

The CPU 31 stores the data regarding the additionally captured image that is captured in the second imaging process (S17) and the data regarding the three-dimensional tomographic image captured in the first imaging process (S1) into a storage device (for example, the NVM 34) in linking to each other. Next, the CPU 31 executes an additionally captured image display control process (S19). In the additionally captured image display control process, the two-dimensional tomographic image 5 extracted from the three-dimensional tomographic image 2 and the additionally captured image stored to be linked to the data regarding the three-dimensional tomographic image 2 are displayed simultaneously or in a switching manner on the monitor 37 together with the data regarding the three-dimensional tomographic image 2 (for example, at least one of the three-dimensional tomographic image 2 and the analysis result for the three-dimensional tomographic image 2). As an example, in the additionally captured image display control process in the present embodiment, the CPU 31 displays a graphical user interface (GUI; for example, an icon) for the user to input an instruction for switching between the extracted two-dimensional tomographic image 5 and the additionally captured image when displaying the data regarding the three-dimensional tomographic image 2 on the monitor 37. When the GUI is operated, the CPU 31 displays the extracted two-dimensional tomographic image 5 and the display of the additionally captured image in a switching manner. In this case, the images linked to each other are smoothly switched, and thus the user's convenience is further improved.

In the related art, even if data is obtained for the same subject eye, data regarding the three-dimensional tomographic image 2 and data regarding an additionally captured image are not stored in a linked state. As a result, even in a viewer that displays an image captured by the OCT apparatus 1 and an analysis result for the image, the data regarding the three-dimensional tomographic image 2 and the data regarding the additionally captured image are handled as completely different data (that is, data showing completely different examination results). In contrast, in the present embodiment, the data regarding the three-dimensional tomographic image 2 and the data regarding the additionally captured image are linked and stored. Therefore, not only on the check screen that allows the user to check an imaging result, but also on the viewer described above, the two-dimensional tomographic image 5 extracted from the three-dimensional tomographic image 2 and the additionally captured image are displayed simultaneously or in a switching manner on the basis of the link together with the data regarding the three-dimensional tomographic image 2.

The techniques disclosed in the above embodiments are merely examples. Therefore, the techniques exemplified in the above embodiments may be changed. For example, only some of the plurality of techniques exemplified in the above embodiments may be executed.

The first imaging process shown in FIG. 4 is an example of the “first imaging step”. The process of extracting and displaying the two-dimensional tomographic image in S6 in FIGS. 4 and S46 in FIG. 7B is an example of the “extraction display step”. The process of setting the additional imaging position in S11 and S12 in FIG. 4 and S44 in FIG. 7B is an example of the “additional imaging position setting step”. The second imaging process shown in S15 to S17 in FIG. 4 is an example of the “second imaging step”. The process of setting the line pattern in S4 and S5 in FIG. 4 is an example of the “line pattern setting step”. The process of setting the additional imaging pattern in S8 and S9 in FIG. 4 is an example of the “additional imaging pattern setting step”. The process of displaying the two-dimensional front image in S31 to S42 in FIG. 7B is an example of the “front image display step”. The process of receiving an instruction from the user in S11 in FIG. 4 is an example of the “position reception step”. The process of displaying the extraction position and the additional imaging position in S6, S9, and S12 in FIG. 4 and S45 in FIG. 7B is an example of the “position display step”. The process of inputting the three-dimensional tomographic image 2 to the mathematical model in S41 in FIG. 7B is an example of the “image input step”. The process of acquiring the certainty factor information in S42 in FIG. 7B is an example of the “certainty factor information acquisition step”.

What is claimed is:

1. An OCT apparatus that processes an OCT signal based on reference light and measurement light with which a subject eye is irradiated to capture a tomographic image of a tissue of the subject eye, the OCT apparatus comprising a controller configured to:
capture a three-dimensional tomographic image of the tissue by irradiating a two-dimensional measurement region, which expands in a direction intersecting an optical axis of the measurement light, with the measurement light;
extract a two-dimensional tomographic image from the three-dimensional tomographic image to display the two-dimensional tomographic image on a display unit;
set an additional imaging position of a tomographic image in a state where the two-dimensional tomographic image is displayed on the display unit; and
perform additional capturing of a tomographic image by irradiating the set additional imaging position with the measurement light.

2. The OCT apparatus according to claim 1,
wherein the controller is further configured to set one of a plurality of types of line patterns, in which at least one of a disposition, a number, and a shape of lines is different from each other, for the two-dimensional measurement region in the three-dimensional tomographic image, and
the controller is configured to extract the two-dimensional tomographic image in a line of the set line pattern from the three-dimensional tomographic image to display the two-dimensional tomographic image on the display unit.

3. The OCT apparatus according to claim 1,
wherein the controller is further configured to set an imaging condition for the additional capturing of the tomographic image, and
the controller is configured to perform the additional capturing of the tomographic image at the additional imaging position according to the set imaging condition.

4. The OCT apparatus according to claim 3,
wherein the imaging condition includes an imaging pattern for the additional capturing of the tomographic image.

5. The OCT apparatus according to claim 1,
wherein the controller is further configured to:
display a two-dimensional front image on the display unit, the two-dimensional front image being an image of the tissue, of which the three-dimensional tomographic image is captured, viewed from a direction along the optical axis of the measurement light; and
receive an instruction from a user for designating a position on the displayed two-dimensional front image, and
the controller is configured to set at least one of an extraction position at which the two-dimensional tomographic image is extracted from the three-dimensional tomographic image and the additional imaging position, in response to the instruction input by the user.

6. The OCT apparatus according to claim 5,
wherein the controller is further configured to display each of the extraction position of the two-dimensional tomographic image and the additional imaging position on the two-dimensional front image.

7. The OCT apparatus according to claim 6,
wherein the controller is configured to:
receive an instruction for designating both the extraction position and the additional imaging position; and
move both the extraction position and the additional imaging position in conjunction with each other on the two-dimensional front image, in response to the instruction input by the user.

8. The OCT apparatus according to claim 5,
wherein the controller is configured to:
receive an instruction from the user for designating a position on the two-dimensional front image that is a still image;
acquire a front observation image of the tissue in real time, viewed from the direction along the optical axis of the measurement light; and
based on the two-dimensional front image and the front observation image, specify the additional imaging position, which is designated on the two-dimensional front image that is a still image, on the front observation image captured in real time to perform the additional capturing of the tomographic image at the specified additional imaging position.

9. The OCT apparatus according to claim 5,
wherein the controller is configured to display an Enface image as the two-dimensional front image on the display unit, the Enface image being an image in which the captured three-dimensional tomographic image is viewed from the direction along the optical axis of the measurement light.

10. The OCT apparatus according to claim 5,
wherein the controller is further configured to analyze the captured three-dimensional tomographic image to generate an analysis map which two-dimensionally represents a distribution of an analysis result, and
the controller is configured to display the analysis map as the two-dimensional front image on the display unit.

11. The OCT apparatus according to claim 5,
wherein the controller is further configured to:
input the captured three-dimensional tomographic image to a mathematical model which is trained by a machine learning algorithm and executes analysis of at least one of a specific structure and a disease of a subject eye captured in an input ophthalmic image; and
acquire certainty factor information indicating a certainty factor of the analysis executed on the input three-dimensional tomographic image by the mathematical model, and
the controller is configured to display the two-dimensional front image including the certainty factor information.

12. The OCT apparatus according to claim 1,
wherein the controller is configured to set at least one of an extraction position at which the two-dimensional tomographic image is extracted from the three-dimensional tomographic image and the additional imaging position, based on an analysis result of the three-dimensional tomographic image.

13. The OCT apparatus according to claim 1,
wherein the controller is further configured to:
store data of the three-dimensional tomographic image of a tissue of a subject eye and data of the tomographic image, which is obtained by performing the additional capturing, of the same tissue of the subject eye into a storage device in linking to each other; and
display the two-dimensional tomographic image extracted from the three-dimensional tomographic image and the tomographic image stored to be linked to the three-dimensional tomographic image simultaneously or in a switching manner together with the data regarding the three-dimensional tomographic image on the display unit.

14. A non-transitory computer-readable storage medium storing an imaging control program executed by an OCT apparatus that processes an OCT signal based on reference light and measurement light with which a subject eye is irradiated to capture a tomographic image of a tissue of the subject eye, the imaging control program comprising instructions which, when the imaging control program is executed by a controller of the OCT apparatus, cause the OCT apparatus to perform:
a first imaging step of capturing a three-dimensional tomographic image of the tissue by irradiating a two-dimensional measurement region, which expands in a direction intersecting an optical axis of the measurement light, with the measurement light;
an extraction display step of extracting a two-dimensional tomographic image from the three-dimensional tomographic image to display the two-dimensional tomographic image on a display unit;
an additional imaging position setting step of setting an additional imaging position of a tomographic image in a state where the two-dimensional tomographic image is displayed on the display unit; and
a second imaging step of performing additional capturing of a tomographic image by irradiating the set additional imaging position with the measurement light.
